(19) United States
(12) Patent Application Publication
(10) Pub. No.: US 2023/0277379 A1
(43) Pub. Date: Sep. 7, 2023

(54) Sleep Mask

(71) Applicant: Aiman Shokr MD, PLLC, Amarillo, TX (US)

(72) Inventor: Aiman Shokr, Amarillo, TX (US)

(21) Appl. No.: 17/684,152

(22) Filed: Mar. 1, 2022

Related U.S. Application Data
(63) Continuation-in-part of application No. 29/828,920, filed on Mar. 1, 2022.

(51) Int. Cl. A61F 9/04

(52) U.S. Cl. CPC A61F 9/04

(57) ABSTRACT

A sleep mask to eliminate shifting during rest, improve comfort for a user and to remain selectively attached to a resting surface. The sleep mask comprises a body and an eye-surround assembly. The body comprises two ends having a first end and a second end. The body comprises a front side, a backside, a top edge, a bottom edge, a body width and a body height. The eye-surround assembly comprises an eye-surround external width, an eye-surround external height, an eye-surround depth and a border portion. The border portion comprises a border width. The resting surface comprises a surface below a head of the user.

### CROSS-REFERENCE TO RELATED APPLICATIONS

Not applicable.

### STATEMENT REGARDING FEDERALLY SPONSORED RESEARCH OR DEVELOPMENT (IF APPLICABLE)

Not applicable.

### REFERENCE TO SEQUENCE LISTING, A TABLE, OR A COMPUTER PROGRAM LISTING COMPACT DISC APPENDIX (IF APPLICABLE)

Not applicable.

### BACKGROUND OF THE INVENTION

No prior art is known to the Applicant

### BRIEF SUMMARY OF THE INVENTION

A sleep mask to eliminate shifting during rest, improve comfort for a user and to remain selectively attached to a resting surface. Said sleep mask comprises a body and an eye-surround assembly. Said body comprises two ends having a first end and a second end. Said body comprises a front side, a backside, a top edge, a bottom edge, a body width and a body height. Said eye-surround assembly comprises an eye-surround external width, an eye-surround external height, an eye-surround depth and a border portion. Said border portion comprises a border width. Said resting surface comprises a surface below a head of said user. Said sleep mask further comprises two end portions. Said body further comprises a central portion having a central portion width comprising a portion between said two end portions. Said sleep mask is configured to further comprise two headstraps. Said two headstraps comprises a first headstrap and a second headstrap. each among said two headstraps comprises a first end attached to a portion of said body, a distal end and a headstrap height. Said two headstraps is configured to each comprise one portion of a headstrap hook-and-look fastener assembly. Said headstrap hook-and-look fastener assembly comprises a first portion and a second portion each chosen among a hook portion or a loop portion. Said headstrap hook-and-look fastener assembly are adapted to selectively mate to one another. each portion of said headstrap hook-and-look fastener assembly are attached to said distal end of each among said two headstraps. for each among said two headstraps, said headstrap height comprises a length sufficient for: wrapping around a top portion of said head of said user, and selectively attaching said two headstraps to one another using said headstrap hook-and-look fastener assembly. Said two headstraps are arranged to begin at a temples of said user and to extend over the top of said head of said user and join to one another over a crown or said top portion of said head.

Said sleep mask to eliminate shifting during rest, improve comfort for said user and to remain selectively attached to said resting surface. Said sleep mask comprises said body and said eye-surround assembly. Said body comprises said two ends having said first end and said second end. Said body comprises said front side, said backside, said top edge, said bottom edge, said body width and said body height. Said eye-surround assembly comprises said eye-surround external width, said eye-surround external height, said eye-surround depth and said border portion. Said border portion comprises said border width. Said resting surface comprises a surface below said head of said user. Said sleep mask further comprises said two end portions. Said body further comprises said central portion having said central portion width comprising a portion between said two end portions.

Said sleep mask to eliminate shifting during rest, improve comfort for said user and to remain selectively attached to said resting surface. Said sleep mask comprises said body and said eye-surround assembly. Said body comprises said two ends having said first end and said second end. Said body comprises said front side, said backside, said top edge, said bottom edge, said body width and said body height. Said eye-surround assembly comprises said eye-surround external width, said eye-surround external height, said eye-surround depth and said border portion. Said border portion comprises said border width. Said resting surface comprises a surface below said head of said user. Said sleep mask further comprises said two end portions. Said body further comprises said central portion having said central portion width comprising a portion between said two end portions. Said two end portions can comprise weighted material configured to hold said two end portions down on said resting surface. Said two end portions comprises a sealed pocket with a soft filler material, such as polystyrene lumps. each among said two end portions comprises an end portion width and an end portion depth. Said sleep mask is configured to rest over said head of said user. Said sleep mask is configured with said central portion being long enough to cover a face of said user extend out to said resting surface. Said two end portions can comprise weighted material configured to hold said two end portions down on said resting surface. Said sleep mask is configured to allow said two end portions to rest on said resting surface. Said sleep mask is adapted to hold itself down while in use by placing said two end portions on said resting surface and extending said central portion of said body across said face of said user.

### BRIEF DESCRIPTION OF THE SEVERAL VIEWS OF THE DRAWING

FIGS. 1A, and 1B illustrate a perspective frontside and backside view of a sleep mask 100.

FIGS. 2A, and 2B illustrate an elevated rear view of said sleep mask 100 and detailed view of an eye-surround assembly 104.

FIG. 3 illustrates a perspective overview of said eye-surround assembly 104 in a detached configuration.

FIGS. 4A and 4B illustrate a perspective overview and elevated side view of a first end portion 112a.

FIG. 5 illustrates a perspective overview of said sleep mask 100 on a user 500.

FIG. 6 illustrates an elevated rear view of said sleep mask 100 with two headstraps 600.

FIG. 7 illustrates a perspective overview of said sleep mask 100 on said user 500.

FIG. 8 illustrates a perspective overview of said user 500 attaching said two headstraps 600 above a head 502.

FIGS. 9A, and 9B illustrate a perspective side view of said user 500 with said sleep mask 100 being unused and in use, respectively.

FIGS. 10A, and 10B illustrate an elevated top view of a resting surface 506 with said user 500, without and with said sleep mask 100, as illustrated.

FIGS. 11A, and 11B illustrate an elevated top view of said resting surface 506 with said user 500, without and with said sleep mask 100, as illustrated.

### DETAILED DESCRIPTION OF THE INVENTION

The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another. It will also be appreciated that such development effort might be complex and time-consuming, but would nevertheless be a routine undertaking for those of ordinary skill in the field of the appropriate art having the benefit of this disclosure. Accordingly, the claims appended hereto are not intended to be limited by the disclosed embodiments, but are to be accorded their widest scope consistent with the principles and features disclosed herein.

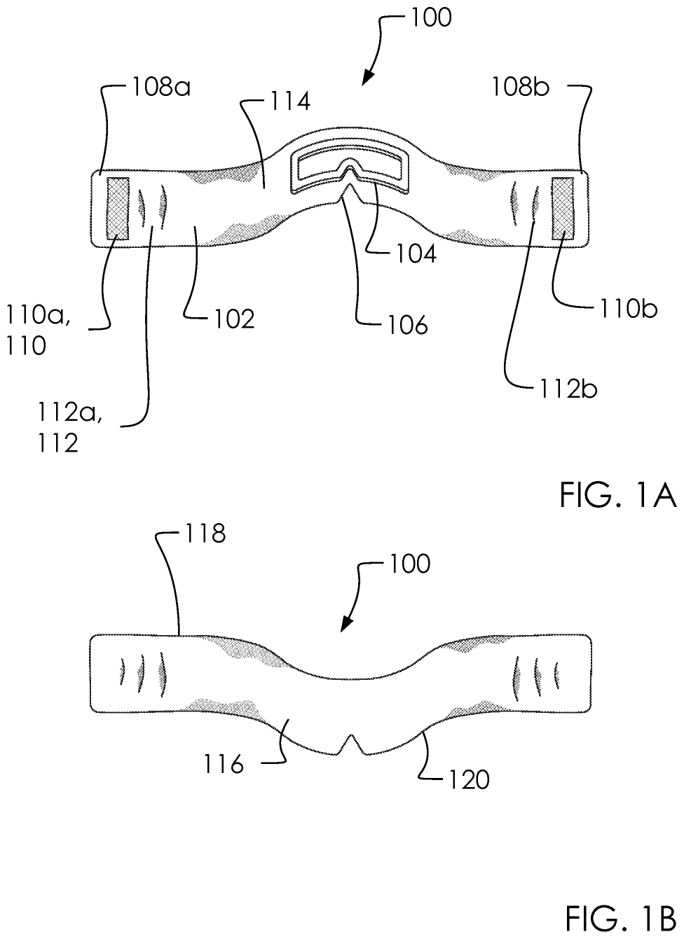

FIGS. 1A, and 1B illustrate a perspective frontside and backside view of a sleep mask 100.

In one embodiment, two hook-and-look fastener portions 110 can comprise a body 102, an eye-surround assembly 104, a nose notch 106, two ends 108 (which can comprise a first end 108a, and a second end 108b), said two hook-and-look fastener portions 110 (which can comprise a first hook-and-look fastener portion 110a, and a second hook-and-look fastener portion 110b), and two end portions 112 (which can comprise a first end portion 112a, and a second end portion 112b).

Said body 102 can comprise a front side 114, a backside 116, a top edge 118, and a bottom edge 120.

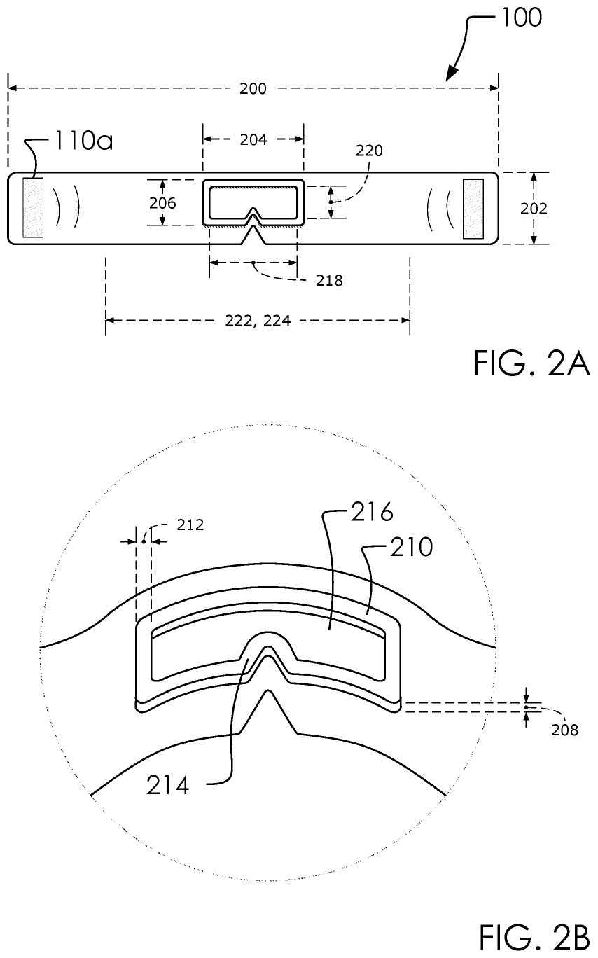

FIGS. 2A, and 2B illustrate an elevated rear view of said sleep mask 100 and detailed view of said eye-surround assembly 104.

In one embodiment, said body 102 can comprise a body width 200 and a body height 202.

In one embodiment, said eye-surround assembly 104 can comprise an eye-surround external width 204, an eye-surround external height 206, an eye-surround depth 208, a border portion 210, a border width 212, a nose notch 214, and an eye-surround cavity 216. In one embodiment, said border portion 210 can comprise a relatively consistent width surrounding said eye-surround cavity 216 being equal to said border width 212. Said eye-surround assembly 104 can further comprise an eye-surround internal width 218, an eye-surround internal height 220, as illustrated.

In one embodiment, said eye-surround external width 204 can comprise 8 inches; said eye-surround external height 206 can comprise 3.5 inches; said eye-surround internal height 220 can comprise 1.5 inches; said eye-surround internal width 218 can comprise 6 inches; and said eye-surround depth 208 can comprise 1 inches.

One feature of said sleep mask 100 can comprise said eye-surround cavity 216 being deep enough to ensure a user's eyelashes and eyes do not feel a pressure from said sleep mask 100.

In one embodiment, said body width 200 can comprise 4 feet; and said body height 202 can comprise 6 inches or more. In one embodiment, the dimensions of said sleep mask 100 can be adapted to ensure that a user's cheeks are covered to assist in providing a complete blackout effect for the user's eyes.

In one embodiment, said two hook-and-look fastener portions 110 can each comprise a single portion of a hook-and-look fastener assembly. In one embodiment, one among or both among said two hook-and-look fastener portions 110 can comprise a hook portion of said hook-and-look fastener assembly.

In one embodiment, said body 102 can comprise a central portion 222 between said two end portions 112. In one embodiment, said central portion 222 can comprise a central portion width 224. In one embodiment, said central portion width 224 can comprise 2 feet.

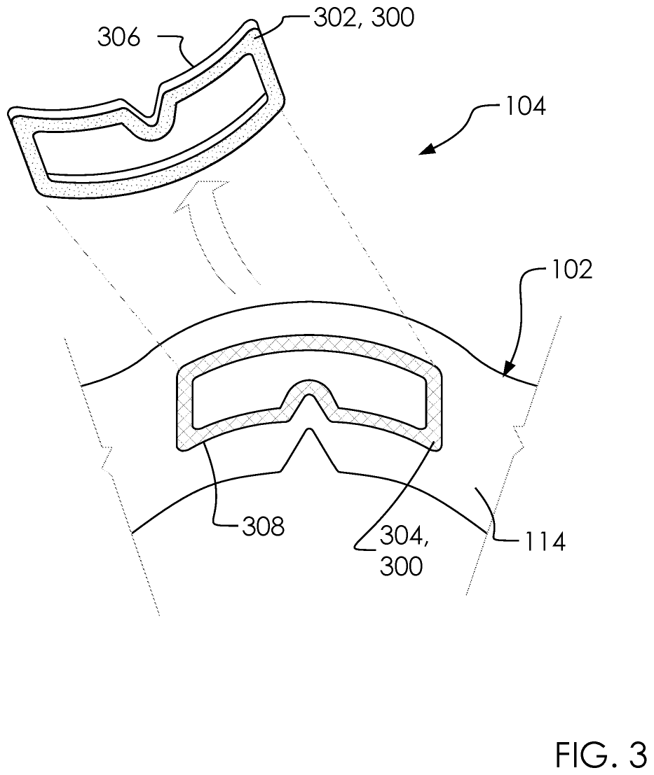

FIG. 3 illustrates a perspective overview of said eye-surround assembly 104 in a detached configuration.

In one embodiment, said eye-surround assembly 104 can be configured to selectively attach to said body 102. In one embodiment, said eye-surround assembly 104 can comprise a hook-and-look fastener assembly 300 which can comprise a first fastener 302 and a second fastener 304. Said eye-surround assembly 104 can comprise an upper portion 306 and a lower portion 308. In one embodiment, said upper portion 306 can comprise said second fastener 304 being attached to said front side 114 of said body 102. In one embodiment, said second fastener 304 and said first fastener 302 can selectively attach to one another, as is known in the art.

In one embodiment, said lower portion 308 can comprise said second fastener 304 attached to a portion of said body 102. Wherein, said upper portion 306 can selectively attach to said lower portion 308 by aligning said first fastener 302 with said second fastener 304 and pressing said hook-and-look fastener assembly 300 together.

In one embodiment, said eye-surround assembly 104 can comprise said upper portion 306 and said lower portion 308 being adapted to selectively release said upper portion 306 from said body 102 for cleaning.

In one embodiment, said second fastener 304 can comprise a loop portion of said hook-and-look fastener assembly 300 and said first fastener 302 can comprise a hook portion of said hook-and-look fastener assembly 300; wherein said sleep mask 100 can be adapted to be used without said upper portion 306 so that said second fastener 304 will not irritate a skin of a user since it is the less abrasive portion of said hook-and-look fastener assembly 300.

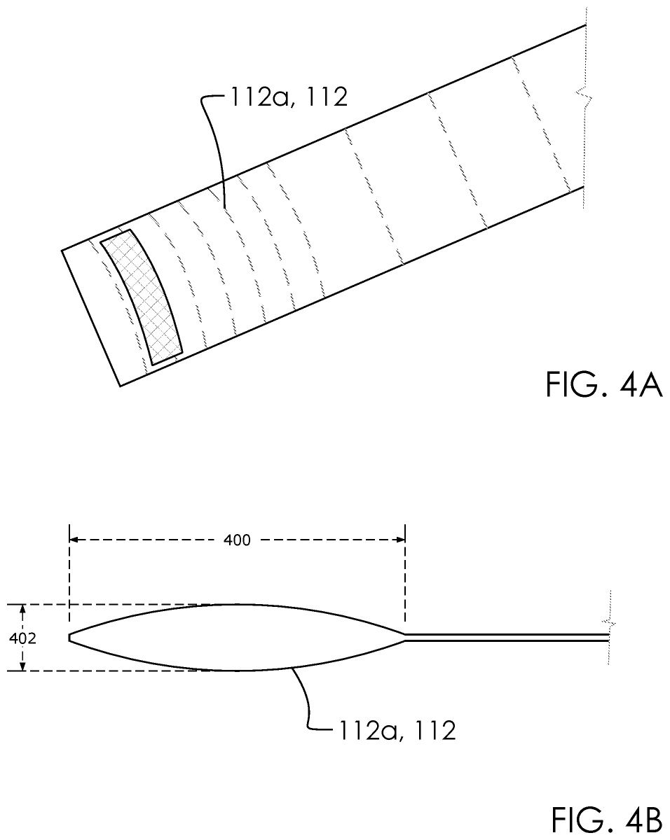

FIGS. 4A and 4B illustrate a perspective overview and elevated side view of said first end portion 112a.

In one embodiment, said two end portions 112 can comprise a sealed pocket with a soft filler material, such as polystyrene lumps commonly used in bean bag chairs. Wherein, said two end portions 112 can comprise a weighted portion of said body 102.

In one embodiment, each among said two end portions 112 can comprise an end portion width 400 and an end portion depth 402. A height of said two end portions 112 can be substantially equal to said body height 202; said end portion width 400 can comprise 12 inches; and said end portion depth 402 can comprise 1-2″. In one embodiment, said body 102 can comprise no filling but-for said two end portions 112.

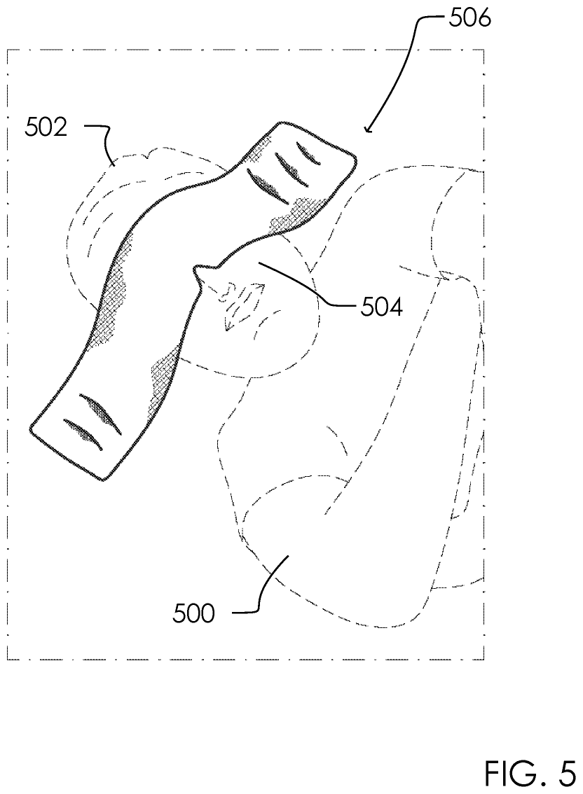

FIG. 5 illustrates a perspective overview of said sleep mask 100 on a user 500.

In one embodiment, said sleep mask 100 can be configured to rest over a head 502 of a user, with said central portion 222 being long enough to cover a face 504 of said user 500 extend out to a resting surface 506 below said head 502 and allow said two end portions 112 to rest on said resting surface 506. Wherein, said sleep mask 100 can be adapted to hold itself down while in use by placing said two end portions 112 on said resting surface 506 and extending said central portion 222 of said body 102 across said face 504 of said user 500.

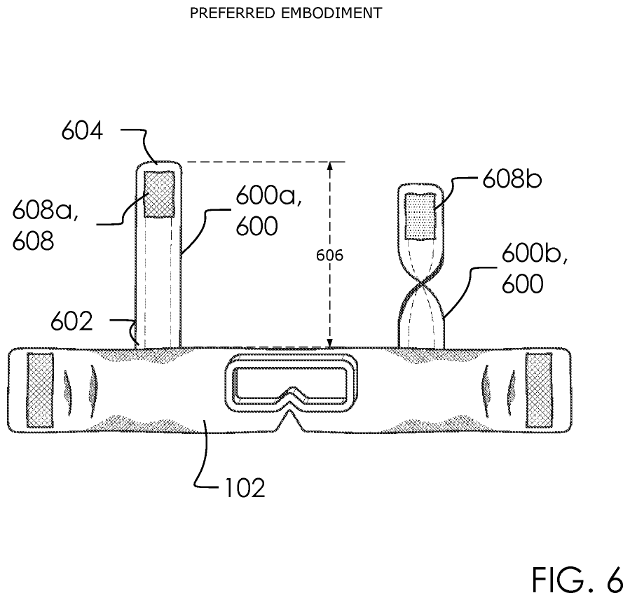

FIG. 6 illustrates an elevated rear view of said sleep mask 100 with two headstraps 600.

Said sleep mask 100 can further comprise said two headstraps 600. Said two headstraps 600 can comprise a first headstrap 600a and a second headstrap 600b.

Each among said two headstraps 600 can comprise a first end 602 attached to a portion of said body 102, a distal end 604 and a headstrap height 606.

In one embodiment, said two headstraps 600 can each comprise one portion of a headstrap hook-and-look fastener assembly 608. Said headstrap hook-and-look fastener assembly 608 can comprise a first portion 608a and a second portion 608b each chosen among a hook portion or a loop portion. Said headstrap hook-and-look fastener assembly 608 can be adapted to selectively mate to one another, as is known in the art. In one embodiment, each portion of said headstrap hook-and-look fastener assembly 608 can be attached to said distal end 604 of each among said two headstraps 600. In one embodiment, said headstrap height 606 can comprise a length sufficient to wrap around a top portion of said head 502 of said user 500, and selectively attach said two headstraps 600 to one another using said headstrap hook-and-look fastener assembly 608, as illustrated below.

In one embodiment, said two headstraps 600 can be attached to said central portion 222 on either side of said eye-surround assembly 104. Said two headstraps 600 can extend up and perpendicular to said body width 200 of said body 102.

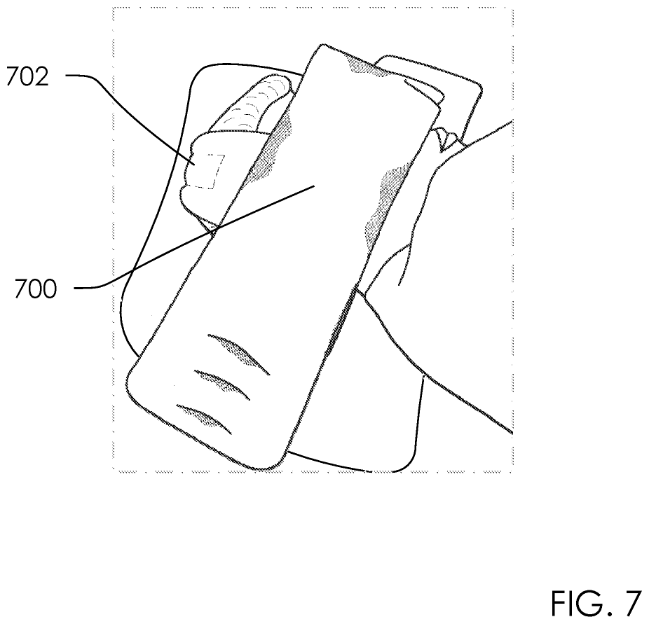

FIG. 7 illustrates a perspective overview of said sleep mask 100 on said user 500.

In one embodiment, said two headstraps 600 can be arranged to begin at a temples 700 of said user 500 and to extend over the top of said head 502 of said user 500 and join to one another over a crown or a top portion 702 of said head 502.

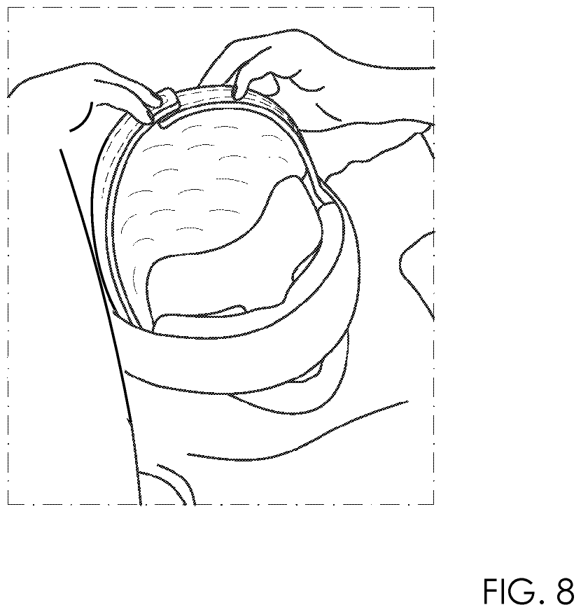

FIG. 8 illustrates a perspective overview of said user 500 attaching said two headstraps 600 above said head 502.

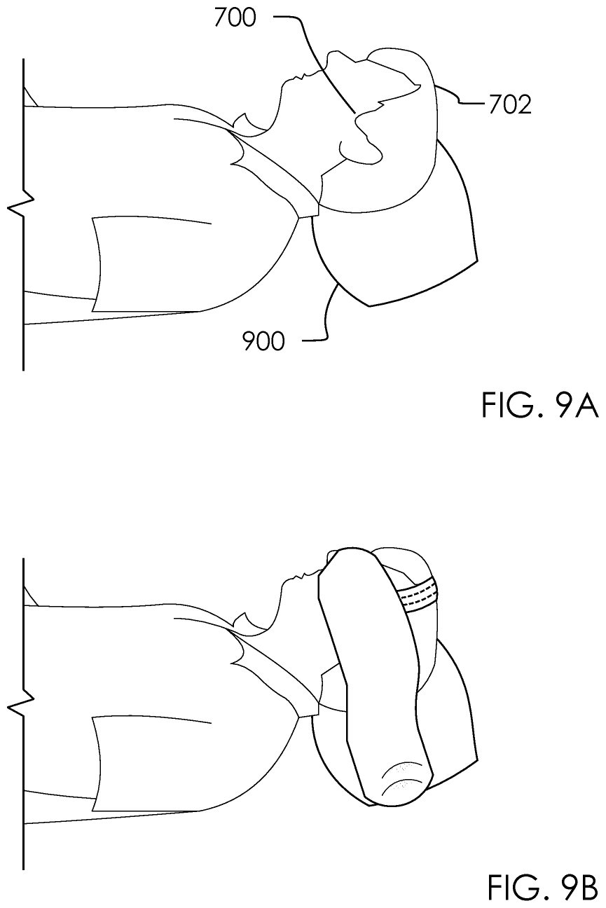

FIGS. 9A, and 9B illustrate a perspective side view of said user 500 with said sleep mask 100 being unused and in use, respectively.

In one embodiment, said sleep mask 100 can be adapted to attach to a pillow, as discussed below.

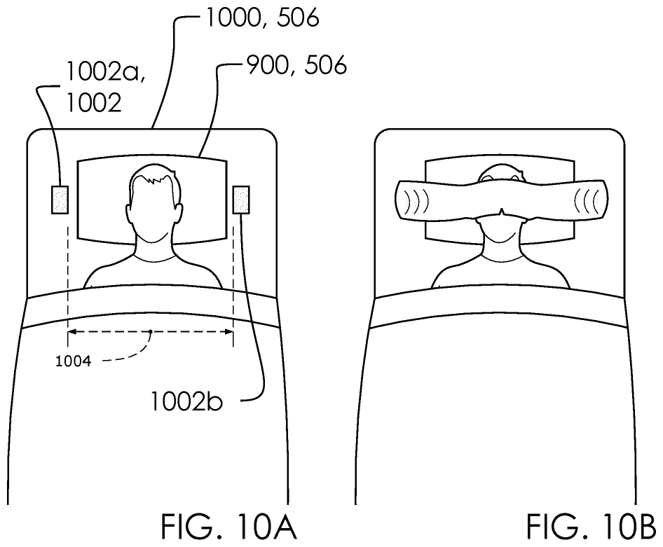

FIGS. 10A, and 10B illustrate an elevated top view of said resting surface 506 with said user 500, without and with said sleep mask 100, as illustrated.

In one embodiment, said resting surface 506 can comprise pillow 900 or a bed 1000, as illustrated.

In one embodiment, said sleep mask 100 can further comprise one or more resting surface hook-and-look fastener mating portions 1002 on said resting surface 506. Said one or more resting surface hook-and-look fastener mating portions 1002 can comprise a first mating portion 1002a and a second mating portion 1002b. Wherein, said one or more resting surface hook-and-look fastener mating portions 1002 can selectively attach with said two hook-and-look fastener portions 110 of said body 102. Further wherein, said sleep mask 100 can be configured to selectively attach to said resting surface 506 to ensure it does not slip while said user 500 is sleeping.

In one embodiment, said one or more resting surface hook-and-look fastener mating portions 1002 can be attached to a top portion of a bed such as a fitted sheet, as illustrated. Wherein, said one or more resting surface hook-and-look fastener mating portions 1002 are arranged at a separation distance 1004 being less than a width between said two hook-and-look fastener portions 110 of said body 102.

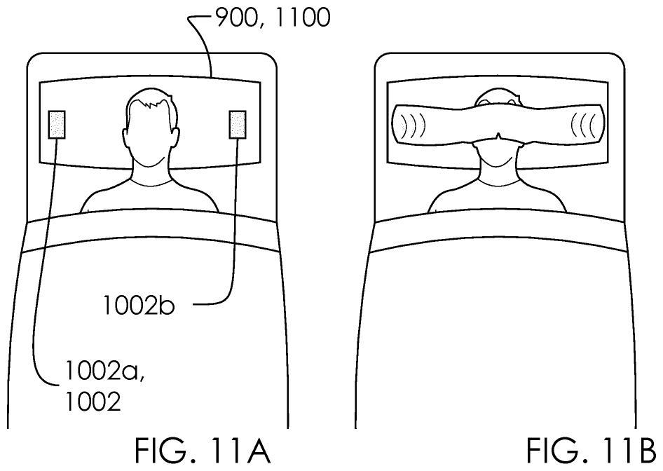

FIGS. 11A, and 11B illustrate an elevated top view of said resting surface 506 with said user 500, without and with said sleep mask 100, as illustrated.

In one embodiment, said one or more resting surface hook-and-look fastener mating portions 1002 can be attached to said pillow 900 or a pillow case 1100. Wherein, said sleep mask 100 is configured to attach to said resting surface 506, such as a portion of said pillow 900.

The following sentences comprise a summary of the claims and may be considered as a preferred embodiment of the current disclosure:

Said sleep mask 100 to eliminate shifting during rest, improve comfort for said user 500 and to remain selectively attached to said resting surface 506. Said sleep mask 100 comprises said body 102 and said eye-surround assembly 104. Said body 102 comprises said two ends 108 having said first end 108a and said second end 108b. Said body 102 comprises said front side 114, said backside 116, said top edge 118, said bottom edge 120, said body width 200 and said body height 202. Said eye-surround assembly 104 comprises said eye-surround external width 204, said eye-surround external height 206, said eye-surround depth 208 and said border portion 210. Said border portion 210 comprises said border width 212. Said resting surface 506 comprises a surface below said head 502 of said user 500. Said sleep mask 100 further comprises said two end portions 112. Said body 102 further comprises said central portion 222 having said central portion width 224 comprising a portion between said two end portions 112. Said sleep mask 100 can be configured to further comprise said two headstraps 600. Said two headstraps 600 comprises said first headstrap 600a and said second headstrap 600b. each among said two headstraps 600 comprises said first end 602 attached to a portion of said body 102, said distal end 604 and said headstrap height 606. Said two headstraps 600 can be configured to each comprise one portion of said headstrap hook-and-look fastener assembly 608. Said headstrap hook-and-look fastener assembly 608 comprises said first portion 608a and said second portion 608b each chosen among a hook portion or a loop portion. Said headstrap hook-and-look fastener assembly 608 can be adapted to selectively mate to one another. each portion of said headstrap hook-and-look fastener assembly 608 can be attached to said distal end 604 of each among said two headstraps 600. for each among said two headstraps 600, said headstrap height 606 comprises a length sufficient for: wrapping around said top portion 702 of said head 502 of said user 500, and selectively attaching said two headstraps 600 to one another using said headstrap hook-and-look fastener assembly 608. Said two headstraps 600 can be arranged to begin at said temples 700 of said user 500 and to extend over the top of said head 502 of said user 500 and join to one another over a crown or said top portion 702 of said head 502.

Said sleep mask 100 to eliminate shifting during rest, improve comfort for said user 500 and to remain selectively attached to said resting surface 506. wherein, said sleep mask 100 comprises said body 102 and said eye-surround assembly 104. Said body 102 comprises said two ends 108 having said first end 108a and said second end 108b. Said body 102 comprises said front side 114, said backside 116, said top edge 118, said bottom edge 120, said body width 200 and said body height 202. Said eye-surround assembly 104 comprises said eye-surround external width 204, said eye-surround external height 206, said eye-surround depth 208 and said border portion 210. Said border portion 210 comprises said border width 212. Said resting surface 506 comprises a surface below said head 502 of said user 500. Said sleep mask 100 further comprises said two end portions 112. Said body 102 further comprises said central portion 222 having said central portion width 224 comprising a portion between said two end portions 112.

Said eye-surround assembly 104 further comprises said nose notch 214. Said sleep mask 100 further comprises said nose notch 106.

Said eye-surround assembly 104 comprises said upper portion 306 and said lower portion 308. Said upper portion 306 of said eye-surround assembly 104 can be configured to selectively attach to said body 102. Said second fastener 304 and said first fastener 302 can be configured to selectively attach to one another. Said sleep mask 100 can be configured to replace or clean said upper portion 306 of said eye-surround assembly 104 by detaching said upper portion 306 for maintenance and reattaching said upper portion 306 with said lower portion 308 of said eye-surround assembly 104.

Said sleep mask 100 comprises said body 102 and said eye-surround assembly 104. Said body 102 comprises said two ends 108 having said first end 108a and said second end 108b. Said body 102 comprises said front side 114, said backside 116, said top edge 118, said bottom edge 120, said body width 200 and said body height 202. Said eye-surround assembly 104 comprises said eye-surround external width 204, said eye-surround external height 206, said eye-surround depth 208 and said border portion 210. Said border portion 210 comprises said border width 212. Said resting surface 506 comprises a surface below said head 502 of said user 500. Said sleep mask 100 further comprises said two end portions 112. Said body 102 further comprises said central portion 222 having said central portion width 224 comprising a portion between said two end portions 112. Said sleep mask 100 can be configured to further comprise said two headstraps 600. Said two headstraps 600 comprises said first headstrap 600a and said second headstrap 600b. each among said two headstraps 600 comprises said first end 602 attached to a portion of said body 102, said distal end 604 and said headstrap height 606. Said two headstraps 600 can be configured to each comprise one portion of said headstrap hook-and-look fastener assembly 608. Said headstrap hook-and-look fastener assembly 608 comprises said first portion 608a and said second portion 608b each chosen among a hook portion or a loop portion. Said headstrap hook-and-look fastener assembly 608 can be adapted to selectively mate to one another. each portion of said headstrap hook-and-look fastener assembly 608 can be attached to said distal end 604 of each among said two headstraps 600. for each among said two headstraps 600, said headstrap height 606 comprises a length sufficient for: wrapping around said top portion 702 of said head 502 of said user 500, and selectively attaching said two headstraps 600 to one another using said headstrap hook-and-look fastener assembly 608. Said two headstraps 600 can be arranged to begin at said temples 700 of said user 500 and to extend over the top of said head 502 of said user 500 and join to one another over a crown or said top portion 702 of said head 502.

Said eye-surround assembly 104 comprises said hook-and-look fastener assembly 300 which comprises said first fastener 302 and said second fastener 304. Said lower portion 308 comprises said second fastener 304 attached to a portion of said body 102. wherein, said upper portion 306 can be configured to selectively attach to said lower portion 308 by aligning said first fastener 302 with said second fastener 304 and pressing said hook-and-look fastener assembly 300 together.

Said second fastener 304 comprises a loop portion of said hook-and-look fastener assembly 300 and said first fastener 302 comprises a hook portion of said hook-and-look fastener assembly 300. wherein said sleep mask 100 can be adapted to be used without said upper portion 306 so that said second fastener 304 will not irritate a skin of a user since it can be the less abrasive portion of said hook-and-look fastener assembly 300

Said two end portions 112 can comprise weighted material configured to hold said two end portions 112 down on said resting surface 506. Said two end portions 112 comprises a sealed pocket with a soft filler material, such as polystyrene lumps. each among said two end portions 112 comprises said end portion width 400 and said end portion depth 402.

A height of said two end portions 112 can be substantially equal to said body height 202. Said end portion width 400 comprises 12 inches. Said end portion depth 402 comprises 1-2″. Said body 102 comprises no filling but-for said two end portions 112.

Said sleep mask 100 can be configured to rest over said head 502 of said user 500. Said sleep mask 100 can be configured with said central portion 222 being long enough to cover said face 504 of said user 500 extend out to said resting surface 506. Said two end portions 112 can comprise weighted material configured to hold said two end portions 112 down on said resting surface 506. Said sleep mask 100 can be configured to allow said two end portions 112 to rest on said resting surface 506. wherein, said sleep mask 100 can be adapted to hold itself down while in use by placing said two end portions 112 on said resting surface 506 and extending said central portion 222 of said body 102 across said face 504 of said user 500.

Said central portion width 224 of said central portion 222 of said body 102 comprises at least 2 feet.

Said sleep mask 100 can be configured to further comprise said two headstraps 600. Said two headstraps 600 comprises said first headstrap 600a and said second headstrap 600b. each among said two headstraps 600 comprises said first end 602 attached to a portion of said body 102, said distal end 604 and said headstrap height 606. Said two headstraps 600 can be configured to each comprise one portion of said headstrap hook-and-look fastener assembly 608. Said headstrap hook-and-look fastener assembly 608 comprises said first portion 608a and said second portion 608b each chosen among a hook portion or a loop portion. Said headstrap hook-and-look fastener assembly 608 can be adapted to selectively mate to one another. each portion of said headstrap hook-and-look fastener assembly 608 can be attached to said distal end 604 of each among said two headstraps 600. for each among said two headstraps 600, said headstrap height 606 comprises a length sufficient for: wrapping around said top portion 702 of said head 502 of said user 500, and selectively attaching said two headstraps 600 to one another using said headstrap hook-and-look fastener assembly 608.

Said two headstraps 600 can be arranged to begin at said temples 700 of said user 500 and to extend over the top of said head 502 of said user 500 and join to one another over a crown or said top portion 702 of said head 502.

Said two headstraps 600 can be attached to said central portion 222 of said body 102 on either side of said eye-surround assembly 104. Said two headstraps 600 can be configured to extend up and perpendicular to said body width 200 of said body 102.

Said sleep mask 100 can be configured to further comprise said one or more resting surface hook-and-look fastener mating portions 1002 on said resting surface 506. Said one or more resting surface hook-and-look fastener mating portions 1002 comprises said first mating portion 1002a and said second mating portion 1002b. Said one or more resting surface hook-and-look fastener mating portions 1002 can be configured to selectively attach with said two hook-and-look fastener portions 110 of said body 102. Said sleep mask 100 can be configured to selectively attach to said resting surface 506 to ensure it does not slip while said user 500 can be sleeping. Said one or more resting surface hook-and-look fastener mating portions 1002 can be arranged at said separation distance 1004 being less than a width between said two hook-and-look fastener portions 110 of said body 102.

Said resting surface 506 comprises said bed 1000. Said one or more resting surface hook-and-look fastener mating portions 1002 can be attached to a top portion of said bed 1000 such as a fitted sheet.

Said one or more resting surface hook-and-look fastener mating portions 1002 can be attached to a portion of said pillow 900. Said sleep mask 100 can be configured to selectively attach to said pillow 900 by: aligning said one or more resting surface hook-and-look fastener mating portions 1002 of said pillow 900 with said two hook-and-look fastener portions 110 of said body 102. selectively adhering said sleep mask 100 with said resting surface 506.

Said body width 200 comprises 4 feet. Said body height 202 comprises 6 inches or more. the dimensions of said sleep mask 100 can be adapted to ensure that a user's cheeks can be covered to assist in providing a complete blackout effect for the user's eyes.

Said sleep mask 100 to eliminate shifting during rest, improve comfort for said user 500 and to remain selectively attached to said resting surface 506. Said sleep mask 100 comprises said body 102 and said eye-surround assembly 104. Said body 102 comprises said two ends 108 having said first end 108a and said second end 108b. Said body 102 comprises said front side 114, said backside 116, said top edge 118, said bottom edge 120, said body width 200 and said body height 202. Said eye-surround assembly 104 comprises said eye-surround external width 204, said eye-surround external height 206, said eye-surround depth 208 and said border portion 210. Said border portion 210 comprises said border width 212. Said resting surface 506 comprises a surface below said head 502 of said user 500. Said sleep mask 100 further comprises said two end portions 112. Said body 102 further comprises said central portion 222 having said central portion width 224 comprising a portion between said two end portions 112. Said two end portions 112 can comprise weighted material configured to hold said two end portions 112 down on said resting surface 506. Said two end portions 112 comprises a sealed pocket with a soft filler material, such as polystyrene lumps. each among said two end portions 112 comprises said end portion width 400 and said end portion depth 402. Said sleep mask 100 can be configured to rest over said head 502 of said user 500. Said sleep mask 100 can be configured with said central portion 222 being long enough to cover said face 504 of said user 500 extend out to said resting surface 506. Said two end portions 112 can comprise weighted material configured to hold said two end portions 112 down on said resting surface 506. Said sleep mask 100 can be configured to allow said two end portions 112 to rest on said resting surface 506. wherein, said sleep mask 100 can be adapted to hold itself down while in use by placing said two end portions 112 on said resting surface 506 and extending said central portion 222 of said body 102 across said face 504 of said user 500.

Said sleep mask 100 can be configured to further comprise said two headstraps 600. Said two headstraps 600 comprises said first headstrap 600a and said second headstrap 600b. each among said two headstraps 600 comprises said first end 602 attached to a portion of said body 102, said distal end 604 and said headstrap height 606. Said two headstraps 600 can be configured to each comprise one portion of said headstrap hook-and-look fastener assembly 608. Said headstrap hook-and-look fastener assembly 608 comprises said first portion 608a and said second portion 608b each chosen among a hook portion or a loop portion. Said headstrap hook-and-look fastener assembly 608 can be adapted to selectively mate to one another. each portion of said headstrap hook-and-look fastener assembly 608 can be attached to said distal end 604 of each among said two headstraps 600. for each among said two headstraps 600, said headstrap height 606 comprises a length sufficient for: wrapping around said top portion 702 of said head 502 of said user 500, and selectively attaching said two headstraps 600 to one another using said headstrap hook-and-look fastener assembly 608.

The following listing of the parts from the figures may be used for the convenience of the reader:
said sleep mask 100,
said body 102,
said eye-surround assembly 104,
said nose notch 106,
said two ends 108,
said first end 108a,
said second end 108b,
said two hook-and-look fastener portions 110,
said first hook-and-look fastener portion 110a,
said second hook-and-look fastener portion 110b,
said two end portions 112,
said first end portion 112a,
said second end portion 112b,
said front side 114,
said backside 116,
said top edge 118,
said bottom edge 120,
said body width 200,
said body height 202,
said eye-surround external width 204,
said eye-surround external height 206,
said eye-surround depth 208,
said border portion 210,
said border width 212,
said nose notch 214,
said eye-surround cavity 216,
said eye-surround internal width 218,
said eye-surround internal height 220,
said central portion 222,
said hook-and-look fastener assembly 300,
said first fastener 302,
said second fastener 304,
said upper portion 306,
said lower portion 308,
said end portion width 400,
said end portion depth 402,
said user 500,
said head 502,
said face 504,
said resting surface 506,
said two headstraps 600,
said first headstrap 600a,
said second headstrap 600b,
said first end 602,
said distal end 604,
said headstrap height 606,
said headstrap hook-and-look fastener assembly 608,
said temples 700,
said top portion 702,
said pillow 900,
said bed 1000,
said one or more resting surface hook-and-look fastener mating portions 1002,
said first mating portion 1002a,
said second mating portion 1002b,
said separation distance 1004 and
said pillow case 1100.

Various changes in the details of the illustrated operational methods are possible without departing from the scope of the following claims. Some embodiments may combine the activities described herein as being separate steps. Similarly, one or more of the described steps may be omitted, depending upon the specific operational environment the method is being implemented in. It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

1. A sleep mask to eliminate shifting during rest, improve comfort for a user and to remain selectively attached to a resting surface; wherein,
said sleep mask comprises a body and an eye-surround assembly;
said body comprises two ends having a first end and a second end;
said body comprises a front side, a backside, a top edge, a bottom edge, a body width and a body height;
said eye-surround assembly comprises an eye-surround external width, an eye-surround external height, an eye-surround depth and a border portion;
said border portion comprises a border width;
said resting surface comprises a surface below a head of said user;
said sleep mask further comprises two end portions;
said body further comprises a central portion having a central portion width comprising a portion between said two end portions;
said sleep mask is configured to further comprise two headstraps;
said two headstraps comprises a first headstrap and a second headstrap;
each among said two headstraps comprises a first end attached to a portion of said body, a distal end and a headstrap height;
said two headstraps is configured to each comprise one portion of a headstrap hook-and-look fastener assembly;
said headstrap hook-and-look fastener assembly comprises a first portion and a second portion each chosen among a hook portion or a loop portion;
said headstrap hook-and-look fastener assembly are adapted to selectively mate to one another;
each portion of said headstrap hook-and-look fastener assembly are attached to said distal end of each among said two headstraps;
for each among said two headstraps, said headstrap height comprises a length sufficient for:
wrapping around a top portion of said head of said user, and
selectively attaching said two headstraps to one another using said headstrap hook-and-look fastener assembly; and
said two headstraps are arranged to begin at a temples of said user and to extend over the top of said head of said user and join to one another over a crown or said top portion of said head.

2. A sleep mask to eliminate shifting during rest, improve comfort for a user and to remain selectively attached to a resting surface; wherein,
said sleep mask comprises a body and an eye-surround assembly;
said body comprises two ends having a first end and a second end;
said body comprises a front side, a backside, a top edge, a bottom edge, a body width and a body height;
said eye-surround assembly comprises an eye-surround external width, an eye-surround external height, an eye-surround depth and a border portion;
said border portion comprises a border width;
said resting surface comprises a surface below a head of said user;
said sleep mask further comprises two end portions; and
said body further comprises a central portion having a central portion width comprising a portion between said two end portions.

3. The sleep mask of claim 2, wherein:
said eye-surround assembly further comprises a nose notch; and
said sleep mask further comprises a nose notch.

4. The sleep mask of claim 2, wherein:
said eye-surround assembly comprises an upper portion and a lower portion;
said upper portion of said eye-surround assembly is configured to selectively attach to said body;
a second fastener and a first fastener are configured to selectively attach to one another; and
said sleep mask is configured to replace or clean said upper portion of said eye-surround assembly by detaching said upper portion for maintenance and reattaching said upper portion with said lower portion of said eye-surround assembly.

5. The sleep mask of claim 4, wherein:
said sleep mask comprises said body and said eye-surround assembly;
said body comprises said two ends having said first end and said second end;
said body comprises said front side, said backside, said top edge, said bottom edge, said body width and said body height;
said eye-surround assembly comprises said eye-surround external width, said eye-surround external height, said eye-surround depth and said border portion;
said border portion comprises said border width;
said resting surface comprises a surface below said head of said user;
said sleep mask further comprises said two end portions;
said body further comprises said central portion having said central portion width comprising a portion between said two end portions;
said sleep mask is configured to further comprise two headstraps;
said two headstraps comprises a first headstrap and a second headstrap;
each among said two headstraps comprises a first end attached to a portion of said body, a distal end and a headstrap height;
said two headstraps is configured to each comprise one portion of a headstrap hook-and-look fastener assembly;
said headstrap hook-and-look fastener assembly comprises a first portion and a second portion each chosen among a hook portion or a loop portion;
said headstrap hook-and-look fastener assembly are adapted to selectively mate to one another;
each portion of said headstrap hook-and-look fastener assembly are attached to said distal end of each among said two headstraps;
for each among said two headstraps, said headstrap height comprises a length sufficient for:
wrapping around a top portion of said head of said user, and
selectively attaching said two headstraps to one another using said headstrap hook-and-look fastener assembly; and
said two headstraps are arranged to begin at a temples of said user and to extend over the top of said head of said user and join to one another over a crown or said top portion of said head.

6. The sleep mask of claim 4, wherein:
said eye-surround assembly comprises a hook-and-look fastener assembly which comprises said first fastener and said second fastener;
said lower portion comprises said second fastener attached to a portion of said body; and
wherein, said upper portion is configured to selectively attach to said lower portion by aligning said first fastener with said second fastener and pressing said hook-and-look fastener assembly together.

7. The sleep mask of claim 6, wherein:
said second fastener comprises a loop portion of said hook-and-look fastener assembly and said first fastener comprises a hook portion of said hook-and-look fastener assembly. wherein said sleep mask is adapted to be used without said upper portion so that said second fastener will not irritate a skin of a user since it is the less abrasive portion of said hook-and-look fastener assembly

8. The sleep mask of claim 2, wherein:
said two end portions can comprise weighted material configured to hold said two end portions down on said resting surface;
said two end portions comprises a sealed pocket with a soft filler material, such as polystyrene lumps; and
each among said two end portions comprises an end portion width and an end portion depth.

9. The sleep mask of claim 8, wherein:
a height of said two end portions is substantially equal to said body height;
said end portion width comprises 12 inches;
said end portion depth comprises 1-2″; and
said body comprises no filling but-for said two end portions.

10. The sleep mask of claim 2, wherein:
said sleep mask is configured to rest over said head of said user;
said sleep mask is configured with said central portion being long enough to cover a face of said user extend out to said resting surface;
said two end portions can comprise weighted material configured to hold said two end portions down on said resting surface;
said sleep mask is configured to allow said two end portions to rest on said resting surface; and
wherein, said sleep mask is adapted to hold itself down while in use by placing said two end portions on said resting surface and extending said central portion of said body across said face of said user.

11. The sleep mask of claim 10, wherein:
said central portion width of said central portion of said body comprises at least 2 feet.

12. The sleep mask of claim 2, wherein:
said sleep mask is configured to further comprise said two headstraps;
said two headstraps comprises said first headstrap and said second headstrap;
each among said two headstraps comprises said first end attached to a portion of said body, said distal end and said headstrap height;
said two headstraps is configured to each comprise one portion of said headstrap hook-and-look fastener assembly;
said headstrap hook-and-look fastener assembly comprises said first portion and said second portion each chosen among a hook portion or a loop portion;
said headstrap hook-and-look fastener assembly are adapted to selectively mate to one another;
each portion of said headstrap hook-and-look fastener assembly are attached to said distal end of each among said two headstraps;
for each among said two headstraps, said headstrap height comprises a length sufficient for:
wrapping around said top portion of said head of said user, and
selectively attaching said two headstraps to one another using said headstrap hook-and-look fastener assembly.

13. The sleep mask of claim 12, wherein:
said two headstraps are arranged to begin at said temples of said user and to extend over the top of said head of said user and join to one another over a crown or said top portion of said head.

14. The sleep mask of claim 12, wherein:
said two headstraps are attached to said central portion of said body on either side of said eye-surround assembly; and
said two headstraps are configured to extend up and perpendicular to said body width of said body.

15. The sleep mask of claim 2, wherein:
said sleep mask is configured to further comprise one or more resting surface hook-and-look fastener mating portions on said resting surface;
said one or more resting surface hook-and-look fastener mating portions comprises a first mating portion and a second mating portion;
said one or more resting surface hook-and-look fastener mating portions are configured to selectively attach with two hook-and-look fastener portions of said body;
said sleep mask is configured to selectively attach to said resting surface to ensure it does not slip while said user is sleeping; and
said one or more resting surface hook-and-look fastener mating portions are arranged at a separation distance being less than a width between said two hook-and-look fastener portions of said body.

16. The sleep mask of claim 15, wherein:
said resting surface comprises a bed; and
said one or more resting surface hook-and-look fastener mating portions are attached to a top portion of said bed such as a fitted sheet.

17. The sleep mask of claim 15, wherein:
said one or more resting surface hook-and-look fastener mating portions are attached to a portion of pillow;
said sleep mask is configured to selectively attach to said pillow by:
aligning said one or more resting surface hook-and-look fastener mating portions of said pillow with said two hook-and-look fastener portions of said body; and
selectively adhering said sleep mask with said resting surface.

18. The sleep mask of claim 2, wherein:
said body width comprises 4 feet; and said body height comprises 6 inches or more;
the dimensions of said sleep mask is adapted to ensure that a user's cheeks are covered to assist in providing a complete blackout effect for the user's eyes.

19. A sleep mask to eliminate shifting during rest, improve comfort for a user and to remain selectively attached to a resting surface; wherein,
said sleep mask comprises a body and an eye-surround assembly;
said body comprises two ends having a first end and a second end;
said body comprises a front side, a backside, a top edge, a bottom edge, a body width and a body height;
said eye-surround assembly comprises an eye-surround external width, an eye-surround external height, an eye-surround depth and a border portion;
said border portion comprises a border width;
said resting surface comprises a surface below a head of said user;
said sleep mask further comprises two end portions;
said body further comprises a central portion having a central portion width comprising a portion between said two end portions;
said two end portions can comprise weighted material configured to hold said two end portions down on said resting surface;
said two end portions comprises a sealed pocket with a soft filler material, such as polystyrene lumps;
each among said two end portions comprises an end portion width and an end portion depth;
said sleep mask is configured to rest over said head of said user;
said sleep mask is configured with said central portion being long enough to cover a face of said user extend out to said resting surface;
said two end portions can comprise weighted material configured to hold said two end portions down on said resting surface;
said sleep mask is configured to allow said two end portions to rest on said resting surface; and
wherein, said sleep mask is adapted to hold itself down while in use by placing said two end portions on said resting surface and extending said central portion of said body across said face of said user.

20. The sleep mask of claim 19, wherein:
said sleep mask is configured to further comprise two headstraps;
said two headstraps comprises a first headstrap and a second headstrap;
each among said two headstraps comprises a first end attached to a portion of said body, a distal end and a headstrap height;
said two headstraps is configured to each comprise one portion of a headstrap hook-and-look fastener assembly;
said headstrap hook-and-look fastener assembly comprises a first portion and a second portion each chosen among a hook portion or a loop portion;
said headstrap hook-and-look fastener assembly are adapted to selectively mate to one another;
each portion of said headstrap hook-and-look fastener assembly are attached to said distal end of each among said two headstraps;
for each among said two headstraps, said headstrap height comprises a length sufficient for:
wrapping around a top portion of said head of said user, and
selectively attaching said two headstraps to one another using said headstrap hook-and-look fastener assembly.
